(19) United States
(12) Patent Application Publication
(10) Pub. No.: US 2020/0249212 A1
(43) Pub. Date: Aug. 6, 2020

(54) COLOR CHANGING GELS AND METHOD OF PRODUCTION

(71) Applicant: Platform Biopolymer Technologies, LLC, Naples, FL (US)

(72) Inventors: Tycho Joseph Speaker, Santa Cruz, CA (US); W. Preston Brawn, Bradenton, FL (US)

(73) Assignee: Platform Biopolymer Technologies, LLC, Naples, FL (US)

(21) Appl. No.: 16/636,248

(22) PCT Filed: Jul. 25, 2018
(86) PCT No.: PCT/US2018/043659

§ 371 Date: Feb. 3, 2020

Related U.S. Application Data
(60) Provisional application No. 62/540,112, filed on Aug. 2, 2017.

(51) Int. Cl. G01N 31/22; G01N 21/80; A61L 9/04

(52) U.S. Cl. CPC G01N 31/221; A61L 9/048; G01N 21/80

(57) ABSTRACT

An aqueous system comprises a pH-responsive visual indicator and a volatile pH modifier. A method of producing a visual signal comprises evaporation of a volatile pH modifier from a system further comprising a pH-responsive visual indicator.

This application claims priority to U.S. Provisional Application No. 62/540,112, filed 2 Aug. 2017, the entire disclosure being incorporated herein by reference in its entirety.

### FIELD OF THE INVENTION

This invention relates generally to aqueous gels for the continuous, controlled release of fragrance, commonly called air treatment gels or air fresheners. It further relates to systems designed to prevent settling or creaming of suspended droplets or particles and to related products and processes for making and using such products. The invention further relates to the interaction between two components to produce a change in color or visible appearance of such gels corresponding to evaporation of one or more components of the gels and signaling the need to replace such consumable products.

### BACKGROUND OF THE INVENTION

The use of aqueous polymer gels as air freshener products is well-established and a substantial commercial market exists for such products. Numerous technologies have been developed to produce such gels, including thermoreversible gelation and chemically induced cross-linking reactions among others. In use, the gel is exposed to air, into which the fragrance components and water content both evaporate over time, delivering the fragrance into the ambient air at a controlled rate. Generally such air freshener products are designed to be useful over a defined time period, for example; one month, or six weeks. At the end of that time period, the rate of fragrance release declines substantially, and the product is considered to be depleted and in need of replacement. However, casual inspection of a depleted product frequently shows that residual gel material, comprising the non-volatile components of the original gel and even some water and fragrance may remain.

Further, some fragrance components may persist for many weeks after the product is considered depleted, but remains if consumers directly smell the packaging. Thus, consumers may mistakenly believe that the product is still functioning properly, even though the product is depleted below levels considered to provide useful function. Hence a need exists for additional cues that indicate the need to replace devices after depletion. Currently some such products include a label or marking area enabling consumers to note the date at which the product is put into use, and guidelines as to how long the product should be used before replacement is required. However, such indirect cues require consumers to perform calculations, read and mark dates, and to remember to check on these passive, often forgotten devices, and are inferior to a direct signal of the need for replacement. Products addressing such problems would be a welcome advance in this industry.

### SUMMARY OF THE INVENTION

The invention provides an aqueous system comprising a pH-responsive visual indicator and a volatile pH modifier.

The pH-responsive visual indicator may comprise Cresol Red, Cresolphthalein (meta), Cresol Purple, Thymol Blue, Methyl Orange—Xylene Cyanol, Bromophenol Blue, Congo Red, Methyl Orange, Alizarin Red S, Bromocresol Green, Dichlorofluorescein, Methyl Red, Bromocresol Purple, Chiorophenol Red, Bromothymol Blue, Phenol Red, Naphtholphthalein (alpha), Phenolphthalein, Cresolphthalein (ortho), Thymolphthalein, or Indigo Carmine, or combinations thereof.

The volatile pH modifier may comprise ammonia, morpholine, and/or water-soluble amines, or combinations thereof.

The volatile pH modifier may evaporate at a rate such that the pH of the system changes by at least 1 pH unit as the system evaporates.

The system may further comprise a fragrance.

The system may further comprise surfactants.

The system may further comprise a solubilized fragrance to form a non-opaque gel.

The system may further comprise a gelling component that solidifies the system.

The system may comprise an air treatment gel.

The invention also provides a method of producing a visual signal comprising evaporation of a volatile pH modifier from a system further comprising a pH-responsive visual indicator.

The method may further comprise dissolving the visual indicator and the volatile pH modifier in an aqueous gel.

The method may further comprise dissolving the visual indicator or the volatile pH modifier or both prior to gelation of the system.

The method may further comprise dissolving the visual indicator or the volatile pH modifier or both after gelation of the system.

### DETAILED DESCRIPTION OF THE INVENTION

The present invention provides a direct and obvious visual indication of depletion of such fragrance gels, that is immediately noticeable from across a room in normal light, and can further provide obvious contrast to packaging or markings, with no special effort or work required upon the part of the consumer. The present invention further provides methods by which such visual changes can be readily included in a range of existing air treatment gel compositions.

The invention provides a composition including a color-changing pH-responsive indicator material and a volatile pH modifier, wherein the pH indicator assumes a visually distinct coloration at an initial concentration of the pH modifier compared to its coloration at a second concentration of the volatile pH modifier after at least partial evaporation of that component, wherein the indicator and pH modifier are included in an aqueous gel. The indicator is present in an amount in a range from 0.001 wt % to 10 wt % relative to the aqueous gel component, and the volatile pH modifier is present in an amount in a range from 0.001 wt % to 10 wt % relative to the aqueous gel component. The invention further provides a method of producing such gels by introducing the indicator and pH modifiers

The invention provides a composition including a succulent extract and an alginic acid component consisting of alginic acid and/or one or more salts thereof, wherein the succulent extract is present in an amount in a range from 0.1 wt % to 1000 wt % relative to the alginic acid component, both on a dry basis.

The inventors formed a cast calcium alginate air treatment gel including a colorant that was also a pH indicator, for easy “tuning” of the gel color, and citric acid and ammonia as convenient pH modifiers used to adjust the final color, in this case a yellow-orange, peach color at approximately pH 5.5 for a peach scented prototype product. After a period of approximately three weeks of ambient air-drying to mimic consumer use and evaluate gel stability during fragrance mass, the inventors were surprised to see that the gel had developed a strong red coloration, noticeably different from the original intended coloration. Without wishing to be bound to a particular explanation, some of the volatile ammonia alkaline component appears to have evaporated, along with the expected loss of water and fragrance, while the non-volatile citric acid component did not volatilize, resulting in a shift to more acidic pH in the remaining gel. The concept of combining a pH-responsive visual indicator with at least one volatile pH modifier is not limited to the calcium alginate system in which it was discovered, but is generalizable to a range of aqueous systems subject to evaporative loss. The key components of such a system comprise an aqueous medium further comprising:
1. a pH-responsive visual indicator dissolved or dispersed in the medium with a resulting vapor pressure less than or equal to that of the water in the medium under the conditions of use, at a level sufficient to be visible
2. a pH modifier dissolved or dispersed in the medium with a vapor pressure greater than that of the visual indicator, at a level sufficient to alter the pH of the aqueous system to provide a visual change in the indicator material

There is no requirement that the aqueous medium be gelled, although the gelled systems comprising air treatment gels are a useful embodiment of the present invention. Generally, where the term “gel” is used, any manner of aqueous systems must be understood to be included, except where air-treatment gels are specifically discussed. There is no requirement as to the order in which the components are added to the medium, and many possible mechanisms of introducing the components to the medium are possible, as will be apparent to persons of ordinary skill in mixing materials and more particularly to forming gelled aqueous systems,

Unless otherwise indicated, all percentages recited herein are on a weight basis.

Visual Indicators with pH-Responsive Characteristics

As used herein, unless otherwise or more precisely specified, the term “pH-responsive visual indicator” refers to materials that demonstrate a visually identifiable change in appearance when dissolved or dispersed in an aqueous system at one pH versus another, different pH. While many pH-responsive visual indicators undergo a shift of their spectroscopic characteristics and visual color, for example, indicator dyes well known in chemistry and other fields, other visual indicators can for instance exhibit a change in solution transparency, for example due to changes in solubility, molecular aggregation or particulate aggregation. Any other visual change induced by a shift in pH can be potentially utilized in the systems of the present invention. Generally, where the term “color-change” is used or reference to changing color is made, any manner of pH-responsive visual change must be understood to be included.

Exemplary color-changing indicators include, but are not restricted to: Cresol Red, Cresolphthalein (meta), Cresol Purple, Thymol Blue, Methyl Orange—Xylene Cyanol, Bromophenol Blue, Congo Red, Methyl Orange, Alizarin Red S, Bromocresol Green, Dichiorofluorescein, Methyl Red, Bromocresol Purple, Chlorophenol Red, Bromothymol Blue, Phenol Red, Naphtholphthalein (alpha), Phenolphthalein, Cresolphthalein (ortho), Thymolphthalein, or Indigo Carmine, or combinations thereof. In some embodiments of the invention combined systems employing more than one visual indicators may be useful.

The pH difference required to produce a detectable change may typically be at least 1.0, 2.0, or 3.0 pH units. It may typically be at most 4.0, 3.0, or 2.0 pH units. In some embodiments, either the beginning or the final pH value may be between 8 and 9, between 7 and 8, between 6 and 7, between 5 and 6, or between 4 and 5.

Suitable Materials for Use as pH Modifiers

Many materials are known to alter the pH of aqueous systems, by a variety of effects including but not limited to direct ionization, ion-scavenging, and through surface effects of materials not dissolved, but rather dispersed in such systems. Any of these materials may be used to adjust the pH of aqueous systems according to the present invention. In embodiments in which the aqueous system is in a gelled state, as for air-treatment gels, it is a requirement that useful pH modifiers not disrupt the gel structure sufficiently to liquefy or degrade the gel. The choice of pH modifier and/or the pH achieved may be restricted to prevent degradation of a dissolved fragrance, color, or other dissolved or dispersed material.

Volatile pH Modifiers

For the purposes of the present invention, the volatile pH modifier must be present in sufficient quantity to induce a change in the pH of the aqueous system sufficient to induce a corresponding visual change in the indicator material. The volatile component must further exhibit sufficient volatility to evaporate from the aqueous system at a rate that causes a visual change or signal associated with the need for replacement or other corrective action. For example, in an air treatment gel, a material with much lower volatility than the fragrance or water components of the gel might eventually evaporate sufficiently to produce a color change, but that visual change might be months or years after the need to replace the gel, rendering it useless for that application. Therefore preferred embodiments of the present invention utilize volatile pH modifiers of volatility from solution near to that of the aqueous phase, for example ammonia and morpholine and/or water-soluble amines, or combinations thereof. Surprisingly, the small quantities of these materials required to shift pH by a useful amount do not result in noticeable malodor in systems of the present invention, despite their distinct odors.

Preparing Compositions of the Invention

Methods of forming color-changing aqueous systems according to the invention will now be described in detail.

Formation of Color-Changing Gels

The essential elements required to form the visually changing gels of the present invention are as follows:
1. a pH-responsive visual indicator is dissolved or dispersed in the medium, at a level sufficient to be visible, producing a coloration or other visible indicator
2. a pH modifier is dissolved or dispersed in the medium with a vapor pressure greater than that of the visual indicator, at a level sufficient to alter the pH of the aqueous system to provide a visual change in the indicator material

The order of addition of the components does not typically affect the products of the invention. If the system further comprises a gelling agent that is heated or strongly influenced by the pH of the system, the indicator and/or the volatile pH modifier can be added to the surface of the gel and distributed into the bulk of the gel by simple diffusion, to avoid interfering with gelation or heat-induced volatilization of the pH modifier component.

The systems of the present invention may further comprise additional polymer or other dissolved or suspended components. It will be obvious to person of ordinary skill in making gelled systems that pH sensitivities or heating requirements may make it desirable to exclude some additional materials or may influence the selection of a particular indicator or pH modifier.

Without being bound to a particular understanding, the properties of the resulting colorized system and the time to visual change are understood to be controlled by multiple factors, including:
1. Selection of the indicator(s)
2. Selection of the volatile pH modifier
3. Concentrations of the indicator and volatile pH modifier component(s)
4. Selection of additional components(s)
5. Concentrations of additional components(s)

Means by Which Indicator(s) and pH Modifier(s) may be Introduced

The visual indicator and pH modifier components can be introduced in various ways, as best suits the particular gel system into which they are introduced. All of the components can be introduced prior to gelation. In other embodiments, one or more of the components can be introduced after gelation is accomplished. For example, a gel system that requires a particular pH to form the gel can be prepared such that the indicator component is included in the gel system before gelation, but the pH modifier component(s) can be added after gelation is accomplished, with spontaneous diffusion of the pH indicators throughout the aqueous gel producing an even distribution of pH after a storage period. A gel system might require heating to liquefy during casting of the gel. As a heated system tends to lose volatile components, the non-volatile indicator and pH modifier can be added prior to casting, and the volatile pH modifier can be added after gelation, to distribute by diffusion.

### EXAMPLES

In some aspects, the invention provides a method of making color-changing aqueous rheological systems. The method comprises combining an pH-responsive indicator with a volatile pH modifier in an aqueous system. When the system is allowed to evaporate, the color will change due to loss of the volatile pH modifier component. In some aspects, the invention provides a product comprising a color-changing aqueous system. In some aspects the invention provides a product comprising an air-treatment gel.

### Example 1

### Formation of a Color-Changing Gel System

An aqueous solution, A, comprising 50 mL of 1.0% wt. sodium alginate (Manucol® DH, FMC Health & Nutrition, Philadelphia, Pa.) was prepared, and adjusted to pH 4.0 by addition of, approximately 0.15 mL of a 10% aqueous solution of citric acid (ESP). To solution A was further added 0.5 mL of a 0.1% solution of methyl red in water, producing a bright cherry red coloration. A second aqueous solution, B, comprising 50 mL of 1.0% wt. Locust Bean Gum (ESP) and further comprising 0.1 g tricalcium citrate (ESP) in suspension was added to solution A while it was stirred at 700 rpm. The system thus formed was immediately poured into a mold and permitted to gel for approximately 3 minutes, forming a non-flowable, solid of red-orange coloration, Immediately following solidification, an aliquot of 0.3 mL of a 9% aqueous ammonia solution was dispensed atop the gel, and the system was covered and stored at room temperature. When examined 8 hours later, the system remained a solid gel, and had assumed an even lemon yellow color throughout. Upon exposure to air, after 15 days of air drying at room temperature, the gel had shrunk substantially and the edges of the gel showed reddening. After 21 days of air drying the gel had further decreased in size and turned a bright red color throughout.

1. An aqueous system comprising a pH-responsive visual indicator and a volatile pH modifier.

2. The system of claim 1 wherein the pH-responsive visual indicator comprises Cresol Red, Cresolphthalein (meta), Cresol Purple, Thymol Blue, Methyl Orange—Xylene Cyanol, Bromophenol Blue, Congo Red, Methyl Orange, Alizarin Red S, Bromocresol Green, Dichlorofluorescein, Methyl Red, Bromocresol Purple, Chlorophenol Red, Bromothymol Blue, Phenol Red, Naphtholphthalein (alpha), Phenolphthalein, Cresolphthalein (ortho), Thymolphthalein, or Indigo Carmine, or combinations thereof.

3. The system of claim 1, wherein the volatile pH modifier comprises ammonia, morpholine, and/or water-soluble amines, or combinations thereof.

4. The system of claim 1, wherein the volatile pH modifier evaporates at a rate such that the pH of the system changes by at least 1 pH unit as the system evaporates.

5. The system of claim 1, further comprising a fragrance.

6. The system of claim 1, further comprising surfactants.

7. The system of claim 1, further comprising a solubilized fragrance to form a non-opaque gel.

8. The system of claim 1, further comprising a gelling component that solidifies the system.

9. The system of claim 1, wherein the system comprises an air treatment gel.

10. A method of producing a visual signal comprising evaporation of a volatile pH modifier from a system further comprising a pH-responsive visual indicator.

11. The method of claim 10, further comprising dissolving the visual indicator and the volatile pH modifier in an aqueous gel.

12. The method of claim 10, further comprising dissolving the visual indicator or the volatile pH modifier or both prior to gelation of the system.

13. The method of claim 10, further comprising dissolving the visual indicator or the volatile pH modifier or both after gelation of the system.
